(19) United States
(12) Patent Application Publication
(10) Pub. No.: US 2009/0163936 A1
(43) Pub. Date: Jun. 25, 2009

(54) Coated Tissue Engineering Scaffold

(76) Inventors: Chunlin Yang, Belle Mead, NJ (US); Wei Kong, Bridgewater, NJ (US)

Correspondence Address: NORRIS MCLAUGHLIN & MARCUS, P.A., 721 ROUTE 202-206, P.O.BOX 5933, BRIDGEWATER, NJ, 08807-5933, US

(21) Appl. No.: 11/962,241

(22) Filed: Dec. 21, 2007

(51) Int. Cl. A61B 17/08

(52) U.S. Cl. 606/151

(57) ABSTRACT

The invention concerns scaffolds comprising a coating on at least one surface that partially penetrates into the void spaces of the scaffold. The invention further concerns scaffolds comprising a partially penetrated anti-adhesion absorbable membrane layer.

### FIELD OF THE INVENTION

The invention relates generally to tissue repair and regeneration and devices for tissue repair and regeneration. The invention concerns scaffolds having coating on at least one surface that partially penetrates into the scaffold structure. In particular, tissue engineering scaffolds having a partially penetrated anti-adhesion coating, i.e., an anti-adhesion absorbable membrane layer to prevent adhesion, on one surface of the tissue engineering scaffold. The tissue engineering scaffold may further have a second coating on other surfaces of the tissue engineering scaffold to guide cell in-growth and enhance tissue integration.

### BACKGROUND OF THE INVENTION

Injuries to tissue, such as musculoskeletal tissue, may require repair by surgical intervention. Such repairs can be affected by suturing the damaged tissue, and/or by mating an implant to the damaged tissue. The implant may provide structural support to the damaged tissue, and it can serve as a substrate upon which cells can grow, thus facilitating healing.

Damage to the abdominal wall is one type of tissue injury which often requires surgical repair. A potentially serious medical condition may occur when the inside layers of the abdominal wall weaken then bulge or tear. The inner lining of the abdomen pushes through the weakened area to form a balloon-like sac. This, in turn, can cause a loop of intestine or abdominal tissue to slip into the sac, causing pain and other potentially serious health problems.

These conditions are usually treated by surgical procedures in which the protruding organs or portions thereof are repositioned. A mesh-like patch in combination with an anti-adhesion barrier is often used to repair the site of the protrusion.

There is continuing need for biocompatible tissue repair implants having sufficient structural integrity to withstand the stresses associated with implantation into an affected area and also possess capability to promote tissue in-growth and integration with in growing tissue, as well as to prevent adhesion. Such biocompatible tissue repair implants are desired for all types of tissue tear repair but in particular for repair of tissue damage to the abdominal wall. Devices, such as tissue engineering scaffolds, with partially penetrated anti-adhesion coatings or membrane layers to prevent adhesion would be particularly desired, including such devices also having a second coating which can guide cell in-growth, enhance tissue integration and provide other therapeutic benefits.

All parts and percentages set forth in this specification and the appended claims are on a weight by weight basis unless specified otherwise.

### BRIEF SUMMARY OF THE INVENTION

The invention pertains to devices, such as scaffolds, which can be applied in surgical procedures to repair tissue damage, such as tissue damage to the abdominal wall. The devices generally have a scaffold, which may be reinforced, and a coating on at least one surface of the scaffold. The coating preferably is an anti-adhesion material, i.e., an anti-adhesion coating. While not wishing to be bound to any theory, the inventors believe that the anti-adhesive properties of the anti-adhesion coating prevents or inhibits bodily organs and/or other internal structures from adhering to wound tissue where the device is implanted. The device may further include one or more separate coatings on the scaffold and scaffold surfaces, i.e., one or more coatings other than the anti-adhesion coating, which provide therapeutic benefits, such as promoting cell in-growth and enhancing tissue integration. These further coatings are preferably on a surface of the scaffold that is not coated with the anti-adhesion material. The device can be further enhanced by bioactives, cells, minced tissue and cell lysates.

In one aspect of the invention, the scaffold has an anti-adhesion coating or layer which partially penetrates into the scaffold structure, such as a partially penetrated absorbable anti-adhesion membrane layer. The anti-adhesion coating or layer provides a barrier to inhibit or prevent internal structure from adhering to the wound tissue where the scaffold is implanted. The anti-adhesion coating or layer is preferably absorbable. In further embodiments of the invention, each surface of the scaffold has a coating, for example a first surface having the anti-adhesion coating partially penetrated into the scaffold structure and a second surface having a layer or coating that guides cell growth and enhances tissue integration.

The scaffold material may be woven or non-woven material. The scaffold may further have reinforcement material which can stabilize the woven or non-woven material, an example of reinforcement material being a mesh. Embodiments of the invention concern absorbable or non-absorbable woven or non-woven material with no mesh, absorbable woven or non-woven material with mesh and non-absorbable woven or non-woven material with mesh.

An embodiment of the invention involves the combination of hydrophilic coating, which may be an absorbable membrane layer and/or an anti-adhesion barrier, having hyaluronic acid, carboxymethyl cellulose (“CMC”), oxidized regenerated celluloses (“ORC”) and combinations thereof, with the scaffold including hydrophobic material. The coating material partially penetrates into the scaffold which completely eliminates or reduces the amount or quantity of glue or film to hold the coating, i.e., the absorbable membrane layer or anti-adhesion barrier, to the scaffold.

### BRIEF DESCRIPTION OF THE SEVERAL VIEWS OF THE DRAWINGS

FIG. 1 is a perspective view of device in accordance with embodiments of the invention having a scaffold structure and an anti-adhesion barrier partially penetrated in the scaffold structure.

FIG. 2 is a magnified perspective view of device shown in FIG. 1 particularly showing the interface between a scaffold structure and an anti-adhesion barrier partially penetrated in the scaffold structure.

FIG. 3 a set of scanning electron microscope (“SEM”) images of mesh reinforced non-woven scaffolds having CMC/ORC coating in accordance with embodiments of the invention.

FIG. 4 is a set of SEM images of mesh reinforced non-woven scaffolds having 1-ethyl-3-(3-dimethylaminopropyl) carbodiimide hydrochloride (“EDC”) cross linked CMC/ORC coating in accordance with embodiments of the invention.

### DETAILED DESCRIPTION OF THE INVENTION

The device has a scaffold having one or more surfaces and a structure having a plurality of fibers with outer surfaces that define one or more, preferably a plurality, of void spaces within the scaffold. Coating material, forming a layer, is on at least one surface of the scaffold and fills at least one void space at or proximate to the surface, preferably all of the void spaces at or proximate to the surface. As such, the coating partially penetrates into the scaffold structure.

In an aspect of the invention the scaffold has an upper surface and/or a lower surface with one or more coatings or layers on the upper surface and/or lower surface. In a further aspect of the invention, one surface of the scaffold is coated with anti-adhesion coating or layer, such as absorbable membrane to prevent adhesion. The absorbable membrane may be partially penetrated into the scaffold structure. The device may also have bioactives, cells, minced tissue and cell lysates either as a component of the scaffold material and/or as part of a coating or layer on one or more of the surfaces of the scaffold, preferably on a surface of the scaffold that does not have the anti-adhesion coating. In an embodiment, the coating on the scaffold surface is an anti-adhesion layer in that it protects internal organs from adhering to the scaffold and/or wound tissue during healing.

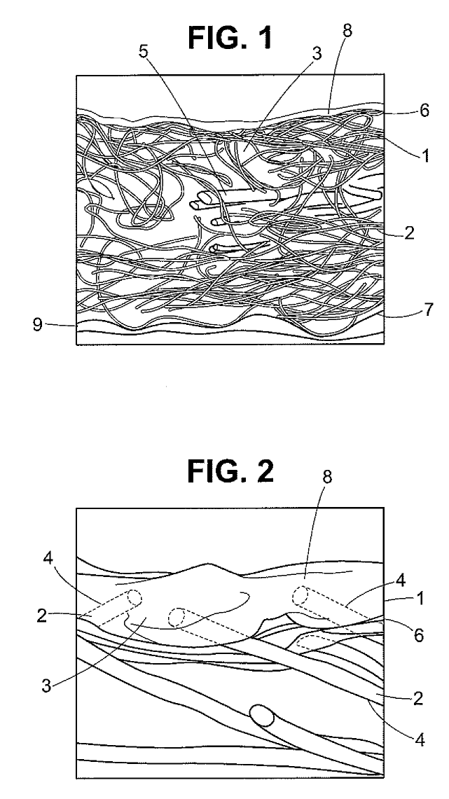

Embodiments of the invention wherein the coating material, such as the absorbable membrane, is integrated with the scaffold by a partial penetration into the scaffold is shown in FIGS. 1 and 2. Referring to FIGS. 1 and 2, the scaffold 1 has fiber structure which in this embodiment has a plurality of fibers 2 and the scaffold 1 has a one or more void spaces 3 within the plurality of fibers 2. In the embodiment of FIGS. 1 and 2 the fibers 2 are in a non-woven structure, it should be understood, however, that the fibers may be woven or non-woven. The fibers 2 have an outer surface 4 and the void spaces 3 are generally defined by the outer surfaces 4 of the fibers 2. The intermingled fibers 2 form gaps which are the void spaces 3 and thus the outer surfaces 4 of the various intermingled fibers 2 defines the one or more void spaces 3 within the scaffold 1.

In further embodiments of the invention, the scaffold is reinforced with a reinforcing material. In the embodiment of FIG. 1, the scaffold has reinforcing material 5 within some or all of the void spaces 3. An example of reinforcing material 5 useful in the invention is a mesh fiber, which provides support to the scaffold structure. ULTRAPRO mesh reinforced polyglactin 910 non-woven scaffold available from Ethicon, Inc. Somerville, N.J., USA (“Ethicon”) may be used for the invention.

As shown in FIGS. 1 and 2, the scaffold 1 generally has an upper surface 6 and a lower surface 7. In the embodiment shown in FIGS. 1 and 2, the device comprises a first coating 8, which may be an anti-adhesion coating, and a second coating 9. Referring to FIG. 1, the first coating 8, such as an absorbable membrane, is at or proximate to the upper surface 6 and the second coating 9, which may be a coating that provides therapeutic benefits such as cell in-growth and enhanced tissue integration, is at or proximate to the lower surface 7. The first coating 8 may include hyaluronic acid, carboxymethyl cellulose (“CMC”), oxidized regenerated celluloses (“ORC”) and combinations thereof. As shown particularly in FIG. 2, the first coating 8, such as an anti-adhesion coating, partially penetrates within the scaffold 1 such that some or all this coating 8 fills at least one void space 3 at or proximate to the upper surface 6 of the scaffold structure and the fibers 2 of the scaffold 1 partially penetrate into the anti-adhesion coating 8. In aspects of the invention, the first coating 8, for example an anti-adhesion coating, fills, as a continuous layer, all of the void spaces 3 at or proximate to the upper surface 6 of the scaffold 1. The coating may cover all of the upper surface of the scaffold, cover substantially all of the upper surface of the scaffold or may cover some of the upper surface of the scaffold. In an embodiment of the invention, the coating 8, typically the anti-adhesion coating, is hydrophilic and the scaffold material is hydrophobic, such as the fibers 2 having a hydrophobic outer surface 4. The combination of the hydrophilic coating material and hydrophobic scaffold material provides for partial penetration of the coating material into the scaffold structure, i.e. into the void spaces, which eliminates the need for glue or a separate film to apply a coating, such as an absorbable membrane and/or anti-adhesion barrier, to the surface of the scaffold.

In an embodiment, the coating material that forms the anti-adhesion coating, such as the absorbable membrane, has hyaluronic acid or CMC, and a combination with ORC, such as INTERCEED® and SURGICEL®, available from Ethicon, Inc. In a further embodiment, the coating material is formed of a combination of hyaluronic acid and CMC with or without ORC. The anti-adhesion coating typically has about 1.5% to about 5%, such as about 2%, hyaluronic acid and/or about 1% to about 10%, such as about 1.5%, CMC, either or both of which may be combined with up to about 5% ORC, such as about 0.1% to about 5% ORC, preferably about 0.5% ORC. The coating may be stabilized by cross linking, such as by EDC, including EDC in a solution containing alcohol, such as ethanol, isopropanol, propanol and combinations thereof, preferably at a concentration between about 50% to about 95%. A preferred cross-linking agent includes an alcohol solution having about 1% EDC, about 10 nM glutaraldehyde and about 0.1% to about 2% divinyl sulfone.

The anti-adhesion coating may be sufficiently thick to adhere to the scaffold structure and provide a barrier to internal organs adhering to the device or wound tissue after implantation. For example, the thickness of the anti-adhesion coating material, such as the absorbable membrane, may be about 5 um to about 250 um. Other coatings on the device may also have a thickness of about 5 um to about 250 um. The depth of penetration of the coating material into the scaffold structure, i.e., the fibers and void space, is preferably about 1 um to about 100 um. Thus, the coating material may penetrate into the void spaces of the fiber web of the scaffold material a distance of about 1 um to about 100 um measured from the upper surface or lower surface of the scaffold, i.e., the surface(s) of the scaffold to which the coating is applied.

In one embodiment of the invention, the scaffold can be formed from a biocompatible polymer. A variety of biocompatible polymers can be used to make the biocompatible tissue implants or scaffold devices according to the invention. The biocompatible polymers can be synthetic polymers, natural polymers or combinations thereof. As used herein the term “synthetic polymer” refers to polymers that are not found in nature, even if the polymers are made from naturally occurring biomaterials. The term “natural polymer” refers to polymers that are naturally occurring. In embodiments where the scaffold includes at least one synthetic polymer, suitable biocompatible synthetic polymers may include polymers selected from the group consisting of aliphatic polyesters, poly(amino acids), poly(propylene fumarate), copoly(ether-esters), polyalkylenes oxalates, polyamides, tyrosine derived polycarbonates, poly(iminocarbonates), polyorthoesters, polyoxaesters, polyamidoesters, polyoxaesters containing amine groups, poly(anhydrides), polyphosphazenes, and blends thereof. Suitable synthetic polymers for use in the invention can also include biosynthetic polymers based on sequences found in collagen, elastin, thrombin, fibronectin, starches, poly(amino acid), gelatin, alginate, pectin, fibrin, oxidized cellulose, chitin, chitosan, tropoelastin, hyaluronic acid, ribonucleic acids, deoxyribonucleic acids, polypeptides, proteins, polysaccharides, polynucleotides and combinations thereof. Non-absorbable biocompatible polymers may also be used for the scaffold, including polyolefins, such as fluorine-containing polyolefins (for example, a mixture of polyvinylidene fluoride and a copolymer of vinylidene fluoride and hexafluoropropylene available from Ethicon, Inc. under the trade name PRONOVA®), polyethylene or polypropylene; polyurethanes; polyesters, such as polyethylene terephthalate or polybutylene terephthalate; and polyamides, also known as nylons, such as nylon-6, nylon-66, or nylon-12.

The scaffold may be woven in the form of felts made of fibers with an average length of about 5 cm and an average diameter of 15 μm needle punched to create the interlock of fibers. The scaffold may also be nonwoven and nonwoven scaffolds in accordance with embodiments are about 1 mm thick and have a density of about 75 mg/cc.

As discussed above, the scaffold may also include a reinforcing material. The reinforcing material may include any absorbable or non-absorbable textile having, for example, woven, knitted, warped knitted (i.e., lace-like), non-woven, and braided structures. In embodiments, the reinforcing material has a mesh-like structure. The mechanical properties of the reinforcing material can be altered by changing the density or texture of the material, the type of knit or weave of the material, the thickness of the material, or by embedding particles in the material. The mechanical properties of the reinforcing material may be altered by creating sites within the reinforcing material, such as a mesh, where the fibers are physically bonded with each other or physically bonded with another agent, such as, for example, an adhesive or a polymer. The reinforcing material can be monofilaments, yarns, threads, braids, or bundles of fibers. These fibers can be made of any biocompatible material including bioabsorbable materials such as polylactic acid (PLA), polyglycolic acid (PGA), polycaprolactone (PCL), polydioxanone (PDO), trimethylene carbonate (TMC), copolymers or blends thereof. The reinforcing material, such as the fibers, can also be made from any biocompatible materials based on natural polymers including silk and collagen-based materials. In embodiments, the fibers can also be made of any biocompatible fiber that is nonresorbable, such as, for example, polyethylene, polyethylene terephthalate, poly(tetrafluoroethylene), polycarbonate, polypropylene, poly(vinyl alcohol) and combinations thereof. In an embodiment, the fibers are formed from 95:5 copolymer of lactide and glycolide.

In a further embodiment, the fibers that form the reinforcing material can be made of a bioabsorbable glass. Bioglass, a silicate containing calcium phosphate glass, or calcium phosphate glass with varying amounts of solid particles added to control resorption time are examples of materials that could be spun into glass fibers and used for the reinforcing material. Suitable solid particles that may be added to the bioabsorbable glass include iron, magnesium, sodium, potassium, and combinations thereof.

In further embodiments, the scaffold can be formed using tissue grafts, such as may be obtained from autogeneic tissue, allogeneic tissue and xenogeneic tissue. By way of non-limiting example, tissues such as skin, cartilage, periosteum, perichondrium, synovium, fascia, mesenter and sinew can be used as tissue grafts to form the biocompatible scaffold. In some embodiments where an allogeneic tissue is used, tissue from a fetus or newborns can be used to avoid the immunogenicity associated with some adult tissues.

One or more bioactive agent(s) may be incorporated within and/or applied to the scaffolds, and/or may be applied to the viable tissue. Preferably, the bioactive agent is incorporated within, or coated on, the scaffold prior to the addition of viable tissue to the scaffold. The bioactive agent may be within the scaffold structure or it may be a coating applied to the surface of the scaffold or a component of the coating material, such as the coatings described herein, for example the absorbable anti-adhesion layer or membrane. The bioactive agent(s) include a variety of effectors that, when present at the site of injury, promote healing and/or regeneration of the affected tissue. In addition to being compounds or agents that promote or expedite healing, the effectors may also include compounds or agents that prevent infection (e.g., antimicrobial agents and antibiotics), compounds or agents that reduce inflammation (e.g., anti-inflammatory agents) and compounds or agents that suppress the immune system (e.g., immunosuppressants).

Other types of effectors that may be present within the device of the invention include heterologous or autologous growth factors, proteins (including matrix proteins), peptides, antibodies, enzymes, platelets, platelet rich plasma, glycoproteins, hormones, cytokines, glycosaminoglycans, nucleic acids, analgesics, viruses, virus particles, and cell types. It is understood that one or more effectors of the same or different functionality may be incorporated within the device. Further, the effectors mentioned herein are non-limiting examples as other effectors as should be understood by one skilled in the art may be included in the device of the invention.

Examples of suitable effectors also include the multitude of heterologous or autologous growth factors known to promote healing and/or regeneration of injured or damaged tissue. These growth factors can be incorporated directly into the scaffold, or alternatively, the scaffold can include a source of growth factors, such as for example, platelets. “Bioactive agents,” as used herein, can include one or more of the following: chemotactic agents; therapeutic agents (e.g., antibiotics, steroidal and non-steroidal analgesics and anti-inflammatories, anti-rejection agents such as immunosuppressants and anti-cancer drugs); various proteins (e.g., short term peptides, bone morphogenic proteins, glycoprotein and lipoprotein), cell attachment mediators, biologically active ligands, integrin binding sequence, ligands, various growth and/or differentiation agents and fragments thereof (e.g., epidermal growth factor (EGF), hepatocyte growth factor (HGF), vascular endothelial growth factors (VEGF), fibroblast growth factors (e.g., bFGF), platelet derived growth factors (PDGF), insulin derived growth factor (e.g., IGF-1, IGF-II) and transforming growth factors (e.g., TGF-β I-III), parathyroid hormone, parathyroid hormone related peptide, bone morphogenic proteins (e.g., BMP-2, BMP-4; BMP-6; BMP-12), sonic hedgehog, growth differentiation factors (e.g., GDF5, GDF6, GDF8), recombinant human growth factors (e.g., MP52), cartilage-derived morphogenic proteins (CDMP-1), small molecules that affect the upregulation of specific growth factors, tenascin-C, hyaluronic acid, chondroitin sulfate, fibronectin, decorin, thromboelastin, thrombin-derived peptides, heparin-binding domains, heparin, heparan sulfate, DNA fragments and DNA plasmids. Suitable effectors likewise include the agonists and antagonists of the agents described above. The growth factor can also include combinations of the growth factors described above. In addition, the growth factor can be autologous growth factor that is supplied by platelets in the blood. The growth factor from platelets may be a cocktail of various growth factors. If other substances have therapeutic value in the orthopedic field, it is anticipated that at least some of these substances will have use in the present invention, and such substances should be included in the meaning of “bioactive agent” and “bioactive agents” unless expressly limited otherwise.

The proteins that may be present within the device, including within the scaffold structure, include proteins that are secreted from a cell or other biological source, such as for example, a platelet, which is housed within the scaffold structure, as well as those that are present within the device in an isolated form. The isolated form of a protein typically is one that is about 55% or greater in purity, i.e., isolated from other cellular proteins, molecules, debris, and the like. In embodiments, the isolated protein is one that is at least about 65% pure, and most preferably one that is at least about 75% to about 95% pure. Notwithstanding the above, one skilled in the art will appreciate that proteins having a purity below about 55% are still considered to be within the scope of the invention. As used herein, the term “protein” embraces glycoproteins, lipoproteins, proteoglycans, peptides, and fragments thereof. Examples of proteins useful as effectors include, but are not limited to, pleiotrophin, endothelin, tenascin, fibronectin, fibrinogen, vitronectin, V-CAM, I-CAM, N-CAM, selectin, cadherin, integrin, laminin, actin, myosin, collagen, microfilament, intermediate filament, antibody, elastin, fibrillin, and fragments thereof.

Viable tissue can also be included in the devices described herein such as being a component of the scaffold structure. The source can vary and the tissue can have a variety of configurations, however, in one embodiment the tissue is in the form of finely minced tissue fragments, which enhance the effectiveness of tissue regrowth and encourage a healing response. In another embodiment, the viable tissue can be in the form of a tissue slice or strip harvested from healthy tissue that contains viable cells capable of tissue regeneration and/or remodeling.

The device may also have cells, such as cells incorporated into the scaffold structure. Suitable cell types that can serve as effectors according to this invention include, but are not limited to, osteocytes, osteoblasts, osteoclasts, fibroblasts, stem cells (such as embryonic stem cells, mesenchymal stem cells and stem cells isolated from adult tissue), pluripotent cells, chondrocyte progenitors, chondrocytes, endothelial cells, macrophages, leukocytes, adipocytes, monocytes, plasma cells, mast cells, umbilical cord cells, placental cells, stromal cells, epithelial cells, myoblasts, tenocytes, ligament fibroblasts, neurons, bone marrow cells, synoviocytes, precursor cells derived from adipose tissue, peripheral blood progenitor cells, genetically transformed cells, a combination of chondrocytes and other cells, a combination of osteocytes and other cells, a combination of synoviocytes and other cells, a combination of bone marrow cells and other cells, a combination of mesenchymal cells and other cells, a combination of stromal cells and other cells, a combination of stem cells and other cells, a combination of embryonic stem cells and other cells, a combination of precursor cells isolated from adult tissue and other cells, a combination of peripheral blood progenitor cells and other cells, a combination of stem cells isolated from adult tissue and other cells, and a combination of genetically transformed cells and other cells. Other cells having therapeutic value, or which may be discovered to have therapeutic use, in the orthopedic field, shall be within the scope of the invention, and such cells should be included within cell or cells that may be incorporated into the device.

The scaffold can also be used in gene therapy techniques in which nucleic acids, viruses, or virus particles, that encode at least one gene product of interest, to specific cells or cell types. Accordingly, the biological effectors can be a nucleic acid (e.g., DNA, RNA, or an oligonucleotide), a virus, a virus particle, or a non-viral vector. The viruses and virus particles may be, or may be derived from, DNA or RNA viruses. In embodiments of the invention, the gene product is selected from the group consisting of proteins, polypeptides, interference ribonucleic acids (iRNA) and combinations thereof.

Once the applicable nucleic acids and/or viral agents (i.e., viruses or viral particles) are incorporated into the scaffold, the device can then be implanted into a particular site to elicit a type of biological response. The nucleic acid or viral agent can then be taken up by the cells and any proteins that they encode can be produced locally by the cells. In one embodiment, the nucleic acid or viral agent can be taken up by the cells within the tissue fragment of the minced tissue suspension, or, in an alternative embodiment, the nucleic acid or viral agent can be taken up by the cells in the tissue surrounding the site of the injured tissue. One skilled in the art will recognize that the protein produced can be a protein of the type noted above, or a similar protein that facilitates an enhanced capacity of the tissue to heal an injury or a disease, combat an infection, or reduce an inflammatory response. Nucleic acids can also be used to block the expression of unwanted gene product that may impact negatively on a tissue repair process or other normal biological processes. DNA, RNA and viral agents are often used to accomplish such an expression blocking function, which is also known as gene expression knock out.

One skilled in the art will appreciate that the identity of the bioactive agent may be determined by a surgeon, based on principles of medical science and the applicable treatment objectives. It is also understood that the bioactive agent or effector can be incorporated within the device, such as the scaffold structure, during, or after manufacture of the device or scaffold structure of the device, or before, during, or after the surgical placement of the device.

The device is made by providing a scaffold and then applying the coating material, preferably in liquid form, and spreading the coating material over at least one surface of the scaffold. The coating is then dried and hardens on the surface of the scaffold to form a membrane or layer on the scaffold surface with partial penetration of void spaces at and/or proximate to the surface or surfaces of the scaffold which interface with the coating, for example as shown in FIGS. 1 and 2 the upper surface 6 of the scaffold.

The following examples are illustrative of the principles and practice of this invention, although not limited thereto. Numerous additional embodiments within the scope and spirit of the invention will become apparent to those skilled in the art once having the benefit of this disclosure.

### EXAMPLES

### Example 1

### Fabrication of Mesh Reinforced Non-Woven Scaffolds

Mesh reinforced 90/10 poly(glycolide-co-lactide) (PGA/PLA) non-woven scaffolds were fabricated. Polypropylene/poliglecaprone-25 mesh sold under the tradename ULTRAPRO (Ethicon) was used as the reinforcing constructs, while 90/10 PGA/PLA nonwoven felts (Ethicon) was the 3D fiber construct. The one nonwoven felt was placed on each side of the mesh and the structure was then needle punched to create the interlock of 90/10 PGA/PLA fibers of the felt with the mesh. The mesh reinforced scaffolds were 1.03 mm thick with a density of 71 mg/cc.

### Example 2

### Fabrication of Mesh Reinforced Non-Woven Scaffolds with a Partially Penetrated Anti-Adhesion Barrier

Mesh reinforced scaffold prepared in Example 1 was coated on one side of the scaffold with a thin coating, i.e. layer or film, of anti-adhesion barrier comprising 1.5% (w/w) CMC and 0.5% (w/w) ORC with and without EDC crosslink. The coated device was prepared as follows. 1.5% (w/w) carboxymethylcellulose (type: 7HFPH, lot: 89726, Hercules, Inc., Wilmington, Del.) solution was first prepared by dissolving 1.5 grams of CMC in 100× grams of water at room temperature. 0.5 grams of oxidized regenerated cellulose (Ethicon) were then mixed into 100 ml CMC solution. A 5×6 cm² mesh reinforced scaffold prepared in Example 1 was placed in a stainless steel stretch frame to provide a flat surface for coating. 3.3 grams of CMC/ORC mixture was spread evenly on one side of the scaffold. The coated scaffold was allowed to air dry overnight and was then cut evenly into two halves. One of the coated scaffolds was crosslinked while the other was not. To crosslink the anti-adhesion barrier, the coated scaffold was incubated with 10 mg/ml EDC in 95% EtOH for 3 hours, washed with 95% EtOH twice, and air dried.

### Example 3

### Fabrication of Biocompatible Bioabsorbable Mesh Reinforced Absorbable Non-Woven Scaffolds with a Partially Penetrated Anti-Adhesion Barrier

Bioabsorbable polydioxanone mesh reinforced 90/10 PGA/PLA non-woven scaffolds are fabricated. Polydioxanone meshes are used as the reinforcing constructs, while 90/10 PGA/PLA felts are the 3D fiber construct. The felts are placed on both sides of the mesh and the structure is then needle punched to create the interlock of 90/10 PGA/PLA fibers of the felts with the mesh. The Polydioxanone mesh reinforced scaffolds are 1.0 mm thick with a density of 70 mg/cc. The Polydioxanone mesh reinforced scaffolds are coated with the CMC/ORC anti-adhesion barrier following the process described in Example 2.

### Example 4

### Fabrication of Biocompatible Absorbable Mesh Reinforced Absorbable Non-Woven Scaffolds with a Partially Penetrated Anti-Adhesion Barrier

Polypropylene/poliglecaprone-25 mesh sold under the tradename ULTRAPRO (Ethicon) reinforced polyethylene terephthalate non-woven scaffolds are fabricated. ULTRAPRO meshes are used as the reinforcing constructs, while non-absorbable polyethylene terephthalate (PET) felts are the 3D fiber construct. The felts are placed on both sides of the mesh and the structure is then needle punched to create the interlock of PET fibers of the felts with the mesh. The ULTRAPRO mesh reinforced scaffolds were 1.0 mm thick with a density of 70 mg/cc. The ULTRAPRO mesh reinforced scaffolds are coated with the CMC/ORC anti-adhesion barrier following the process described in Example 2.

### Example 5

### SEM Evaluation

The samples of the coated scaffold prepared in accordance with Example 2 were mounted on a microscope stud and coated with a thin layer of gold using the EMS 550 sputter coater. SEM analysis was performed using the JEOL JSM-5900LV SEM. The surfaces and cross-sectional areas were examined for each sample. The SEM showed a CMC/ORC coated outer layer of the non-woven/mesh composite.

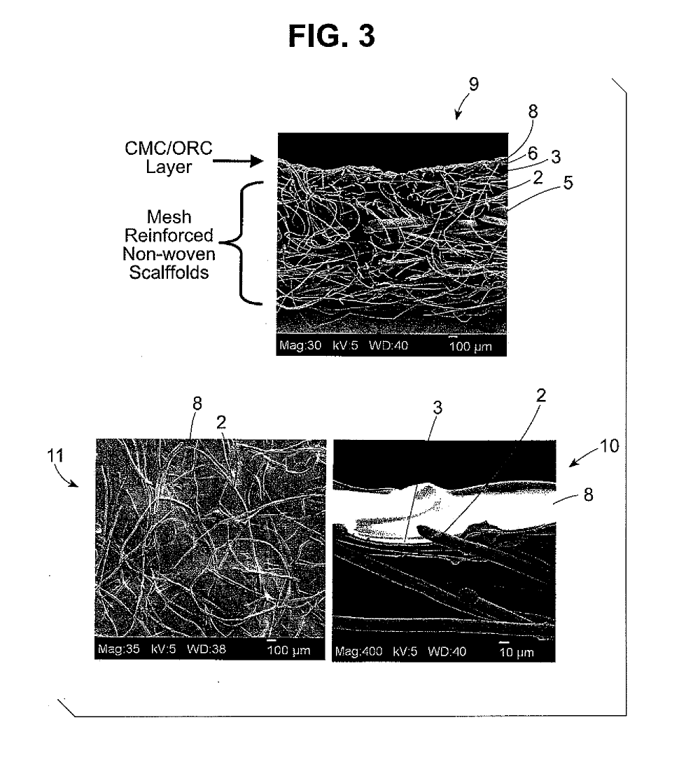

FIG. 3 shows SEM images of CMC/ORC coated ULTRAPRO mesh reinforced vicryl non-woven scaffolds (without crosslinking). SEM image 9 shows an anti-adhesion coating 8 partially penetrating within the scaffold structure such that some or all the anti-adhesion coating 8 fills at least one void space 3 at or proximate to the upper surface 6 of the scaffold structure and the fibers 2 of the scaffold partially penetrate into the anti-adhesion coating 8. The interaction between the fibers 2, void spaces 3 and anti-adhesion coating 8 is shown in greater detail in SEM image 10 of FIG. 3 which is a 400× magnification of a part of the cross-sectional view of the device shown in SEM image 9 of FIG. 3. In SEM Image 10, the anti-adhesion coating 8 is shown filling a void space 3 with the fibers 2 penetrating into the anti-adhesion coating. The SEM image 11 of FIG. 3 shows the upper surface of the scaffold having the anti-adhesion coating 8 with the fibers 2.

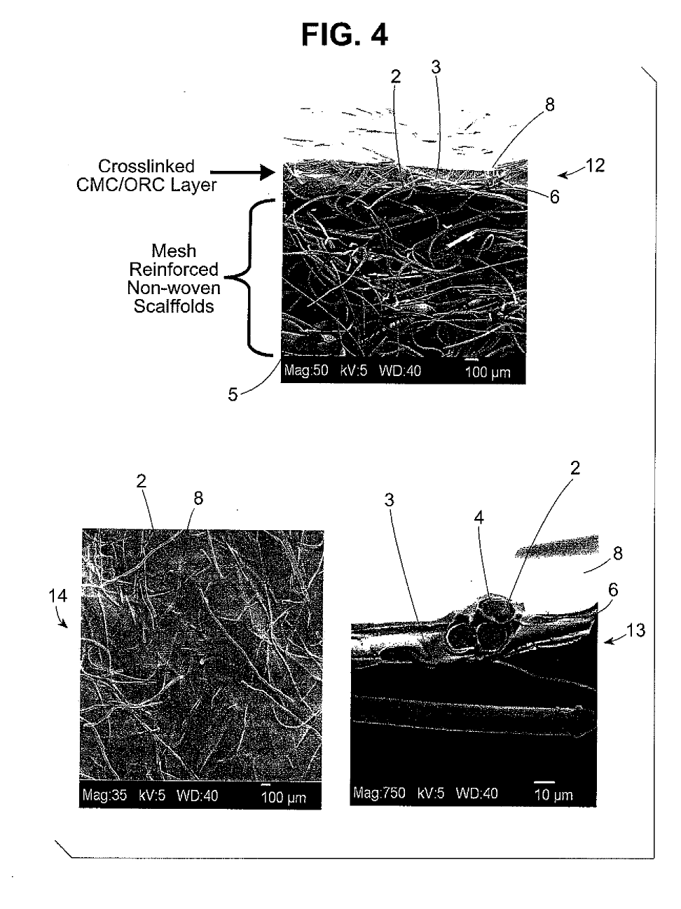

FIG. 4 shows SEM images of EDC cross linked CMC/ORC coated ULTRAPRO mesh reinforced vicryl non-woven scaffolds. SEM image 12 shows an anti-adhesion coating 8 partially penetrating within the scaffold structure such that some or all the anti-adhesion coating 8 fills at least one void space 3 at or proximate to the upper surface 6 of the scaffold structure and the fibers 2 of the scaffold partially penetrate into the anti-adhesion coating 8. The interaction between the fibers 2, void spaces 3 and anti-adhesion coating 8 is shown in greater detail in SEM image 13 of FIG. 4 which is a 750× magnification of a part of the cross-sectional view of the device shown in SEM image 12 of FIG. 4. In SEM Image 13, the anti-adhesion coating 8 is shown filling a void space 3 with the fibers 2 penetrating into the anti-adhesion coating. SEM image 13 also shows the outer surface 4 of the fibers 2 and the interface of the anti-adhesion coating 10 with the outer surfaces 4. The SEM image 14 of FIG. 4 shows the upper surface of the scaffold having the anti-adhesion coating 8 with the fibers 2.

### Example 6

### Rabbit Sidewall Adhesion Model Study

A midline laparotomy was performed. The cecum and bowel was exteriorized and digital pressure exerted to create subserosal hemorrhages over all surfaces. The damaged intestine was lightly abraded with 4″×4″ 4-ply sterile gauze until punctate bleeding was observed. The cecum and bowel were then returned to their normal anatomic position. A 5×3 cm² area of peritoneum and transversus abdominous muscle was removed on the right lateral abdominal wall to create a defect. Then test material, cross linked CMC/ORC coated ULTRAPRO Mesh reinforced polyglactin 910 non-woven scaffolds, as prepared in Example 2, was applied to the defect using suture technique. The surgical controls received no test material. The abdominal wall and skin was closed in a standard manner.

All the controls showed adhesion of the abraded cecum to the sidewall defects. It was observed that all three animals treated with cross linked CMC/ORC coated ULTRAPRO Mesh reinforced polyglactin 910 non-woven scaffolds did not show any adhesion of abraded cecum to the sidewall defect.

### Example 7

### Coating Mesh Reinforced Non-Woven Scaffolds with Different Concentrations of CMC

Mesh reinforced 90/10 PGA/PLA non-woven scaffold, as prepared in Example 1, was coated on one side of the scaffold with a thin coating, i.e., layer or film, of anti-adhesion barrier comprising 5 different concentrations of CMC. The coated device was prepared as follows. 0.5, 1.0, 2.5, 5.0 and 10 mg/ml CMC (type: 7HFPH, lot: 77146, Hercules, Inc., Wilmington, Del.) solutions were prepared at room temperature. Each solution was coated onto one side of the mesh reinforced scaffold that was stretched flat in a stainless steel stretch frame. The coated scaffolds were allowed to air dry overnight. The coated scaffolds were evaluated by scanning electron microscopy (SEM) as described in Example 5. It was found that CMC formed a sufficiently intact layer at a concentration of 10 mg/ml.

Although this invention has been shown and described with respect to detailed embodiments thereof, it will be understood by those skilled in the art that various changes in form and detail thereof may be made without departing from the spirit and scope of the claimed invention.

What is claimed is:

1. A device comprising
a) a scaffold having one or more surfaces the scaffold comprising a plurality of fibers with outer surfaces and one or more void spaces defined by the outer surfaces of the fibers; and
b) a coating on at least one surface of the scaffold
wherein the coating fills at least one void space.

2. The device of claim 1 wherein the fibers are hydrophobic and the coating is hydrophilic.

3. The device of claim 1 wherein the coating comprises a material selected from the group consisting of hyaluronic acid, carboxymethylcellulose and combinations thereof.

4. The device of claim 3 wherein the coating further comprises oxidized regenerated celluloses.

5. The device of claim 3 wherein the material is stabilized by cross linking.

6. The device of claim 5 wherein the material is cross-linked by 1-ethyl-3-(3-dimethylaminopropyl) carbodiimide hydrochloride.

7. The device of claim 1 wherein the scaffold further comprises reinforcing material.

8. The device of claim 7 wherein the reinforcing material is an absorbable or nonabsorbable textile.

9. The device of claim 8 wherein the reinforcing material comprises a bioabsorbable material selected from the group consisting of polylactic acid (PLA), polyglycolic acid (PGA), polycaprolactone (PCL), polydioxanone (PDO), trimethylene carbonate (TMC), copolymers and combinations thereof.

10. The device of claim 9 wherein the bioabsorbable material is a copolymer of lactide and glycolide.

11. The device of claim 7 wherein the reinforcing material comprises biocompatible materials based on natural polymers.

12. The device of claim 7 wherein the reinforcing material comprises a nonresorbable biocompatible fiber selected from the group consisting of polyethylene, polyethylene terephthalate, poly(tetrafluoroethylene), polycarbonate, polypropylene, poly(vinyl alcohol) and combinations thereof.

13. The device of claim 7 wherein the reinforcing material comprises bioabsorbable glass.

14. The device of claim 1 wherein the coating provides an anti-adhesion barrier.

15. The device of claim 1 wherein the coating has a thickness of about 5 um to about 250 um.

16. The device of claim 1 wherein the coating penetrates into the void spaces of the scaffold about 1 um to about 100 um from the surface of the scaffold to which the coating is applied.

17. The device of claim 1 wherein the scaffold comprises a biocompatible polymer.

18. The device of claim 17 wherein the biocompatible polymer is selected from the group consisting of aliphatic polyesters, poly(amino acids), poly(propylene fumarate), copoly(ether-esters), polyalkylenes oxalates, polyamides, tyrosine derived polycarbonates, poly(iminocarbonates), polyorthoesters, polyoxaesters, polyamidoesters, polyoxaesters containing amine groups, poly(anhydrides), polyphosphazenes, biosynthetic polymers based on sequences found in collagen, elastin, thrombin, fibronectin, starches, poly(amino acid), gelatin, alginate, pectin, fibrin, oxidized cellulose, chitin, chitosan, tropoelastin, hyaluronic acid, ribonucleic acids, deoxyribonucleic acids, polypeptides, proteins, polysaccharides, polynucleotides polyolefins, polyurethanes, polyesters, polyamides and combinations thereof.

19. The device of claim 1 wherein the scaffold is formed using tissue grafts.

20. The device of claim 1 further comprising one or more bioactive agents.

21. The device of claim 1 further comprising viable tissue.

22. The device of claim 1 comprising cells incorporated into the scaffold structure.

23. The device of claim 23 wherein the cells are selected from the group consisting of osteocytes, osteoblasts, osteoclasts, fibroblasts, stem cells, pluripotent cells, chondrocyte progenitors, chondrocytes, endothelial cells, macrophages, leukocytes, adipocytes, monocytes, plasma cells, mast cells, umbilical cord cells, placental cells, stromal cells, epithelial cells, myoblasts, tenocytes, ligament fibroblasts, neurons, bone marrow cells, synoviocytes, precursor cells derived from adipose tissue, peripheral blood progenitor cells, genetically transformed cells, precursor cells isolated from adult tissue and combinations thereof.

24. The device of claim 1 further comprising a biological effector for use in gene therapy techniques.

25. The device of claim 24 wherein the biological effector is selected from the group consisting of nucleic acid, virus, virus particle and non-viral vector.
